SEQUENCE LISTING (34 sequences)

SEQ ID NO: 1 - DNA, 34 bp, Artificial Sequence
  Human Factor VIII derivatives
ggaaccatcgccagaagtccttggaaatctcgcc

SEQ ID NO: 2 - DNA, 34 bp, Artificial Sequence
  Human Factor VIII derivatives
ggcgagatttccaaggacttctggcgatggttcc

SEQ ID NO: 3 - DNA, 32 bp, Artificial Sequence
  Human Factor VIII derivatives
cccaccaacatggtggcatggaagcttatgtc

SEQ ID NO: 4 - DNA, 32 bp, Artificial Sequence
  Human Factor VIII derivatives
gacataagcttccatgccaccatgttggtggg

SEQ ID NO: 5 - DNA, 47 bp, Artificial Sequence
  Human Factor VIII derivatives
cagaggaaccccaactacgacgtaaaaataatgaagaagcggaagac

SEQ ID NO: 6 - DNA, 47 bp, Artificial Sequence
  Human Factor VIII derivatives
gtcttccgcttcttcattatttttacgtcgtagttggggttcctctg

SEQ ID NO: 7 - DNA, 42 bp, Artificial Sequence
  Human Factor VIII derivatives
cccaactacgaatgaaaaatgatgaagaagcggaagactatg

SEQ ID NO: 8 - DNA, 42 bp, Artificial Sequence
  Human Factor VIII derivatives
catagtcttccgcttcttcatcatttttcattcgtagttggg

SEQ ID NO: 9 - DNA, 40 bp, Artificial Sequence
  Human Factor VIII derivatives
gaagaagcggaagactatgatgataatcttactgattctg

SEQ ID NO: 10 - DNA, 40 bp, Artificial Sequence
  Human Factor VIII derivatives
cagaatcagtaagattatcatcatagtcttccgcttcttc

SEQ ID NO: 11 - DNA, 38 bp, Artificial Sequence
  Human Factor VIII derivatives
ggtcaggtttgatgatgacgactctccttcctttatcc

SEQ ID NO: 12 - DNA, 38 bp, Artificial Sequence
  Human Factor VIII derivatives
ggataaaggaaggagagtcgtcatcatcaaacctgacc

SEQ ID NO: 13 - DNA, 38 bp, Artificial Sequence
  Human Factor VIII derivatives
cccttagtcctcgccccctctgacagaagttataaaag

SEQ ID NO: 14 - DNA, 38 bp, Artificial Sequence
  Human Factor VIII derivatives
cttttataacttctgtcagagggggcgaggactaaggg

SEQ ID NO: 15 - DNA, 41 bp, Artificial Sequence
  Human Factor VIII derivatives
gtccgatttatggcatacacagatgttacctttaagactcg

SEQ ID NO: 16 - DNA, 41 bp, Artificial Sequence
  Human Factor VIII derivatives
cgagtcttaaaggtaacatctgtgtatgccataaatcggac

SEQ ID NO: 17 - DNA, 37 bp, Artificial Sequence
  Human Factor VIII derivatives
cctttaagactcgtaaagctattcagcatgaatcagg

SEQ ID NO: 18 - DNA, 37 bp, Artificial Sequence
  Human Factor VIII derivatives
cctgattcatgctgaatagctttacgagtcttaaagg

SEQ ID NO: 19 - DNA, 46 bp, Artificial Sequence
  Human Factor VIII derivatives
cacactgttgattatatttaagaataaagcaagcagaccatataac

SEQ ID NO: 20 - DNA, 46 bp, Artificial Sequence
  Human Factor VIII derivatives
gttatatggtctgcttgctttattcttaaatataatcaacagtgtg

SEQ ID NO: 21 - DNA, 40 bp, Artificial Sequence
  Human Factor VIII derivatives
ccctcacggaatcactgatgtctctcctttgtattcaagg

SEQ ID NO: 22 - DNA, 40 bp, Artificial Sequence
  Human Factor VIII derivatives
ccttgaatacaaaggagagacatcagtgattccgtgaggg

SEQ ID NO: 23 - DNA, 38 bp, Artificial Sequence
  Human Factor VIII derivatives
gatgtccgtcctttgtattcagggagattaccaaaagg

SEQ ID NO: 24 - DNA, 38 bp, Artificial Sequence
  Human Factor VIII derivatives
ccttttggtaatctccctgaatacaaaggacggacatc

SEQ ID NO: 25 - DNA, 36 bp, Artificial Sequence
  Human Factor VIII derivatives
ctgtatttgatgagaaccaaagctggtacctcacag

SEQ ID NO: 26 - DNA, 36 bp, Artificial Sequence
  Human Factor VIII derivatives
ctgtgaggtaccagctttggttctcatcaaatacag

SEQ ID NO: 27 - DNA, 30 bp, Artificial Sequence
  Human Factor VIII derivatives
ctccccaatccagatggagtgcagcttgag

SEQ ID NO: 28 - DNA, 30 bp, Artificial Sequence
  Human Factor VIII derivatives
ctcaagctgcactccatctggattggggag

SEQ ID NO: 29 - DNA, 32 bp, Artificial Sequence
  Human Factor VIII derivatives
cagctggagtgcagcttcaggatccagagttc

SEQ ID NO: 30 - DNA, 32 bp, Artificial Sequence
  Human Factor VIII derivatives
gaactctggatcctgaagctgcactccagctg

SEQ ID NO: 31 - DNA, 49 bp, Artificial Sequence
  Human Factor VIII derivatives
cgatggtatctgctcagcatgaagagcaatgaaaacatccattctattc

SEQ ID NO: 32 - DNA, 49 bp, Artificial Sequence
  Human Factor VIII derivatives
gaatagaatggatgttttcattgctcttcatgctgagcagataccatcg

SEQ ID NO: 33 - protein, 2114 aa, Porcine
AIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASQKQFQTITSPEDDVELDPQSGERTQALEELSVPSGDGSMLLGQNPAPHGSSSSDLQEARNEADDYLPGARERNTAPSAAARLRPELHHSAERVLTPEPEKELKKLDSKMSSSSDLLKTSPTIPSDTLSAETERTHSLGPPHPQVNFRSQLGAIVLGKNSSHFIGAGVPLGSTEEDHESSLGENVSPVESDGIFEKERAHGPASLTKDDVLFKVNISLVKTNKARVYLKTNRKIHIDDAALLTENRASATFMDKNTTASGLNHVSNWIKGPLGKNPLSSERGPSPELLTSSGSGKSVKGQSSGQGRIRVAVEEEELSKGKEMMLPNSELTFLTNSADVQGNDTHSQGKKSREEMERREKLVQEKVDLPQVYTATGTKNFLRNIFHQSTEPSVEGFDGGSHAPVPQDSRSLNDSAERAETHIAHFSAIREEAPLEAPGNRTGPGPRSAVPRRVKQSLKQIRLPLEEIKPERGVVLNATSTRWSESSPILQGAKRNNLSLPFLTLEMAGGQGKISALGKSAAGPLASGKLEKAVLSSAGLSEASGKAEFLPKVRVHREDLLPQKTSNVSCAHGDLGQEIFLQKTRGPVNLNKVNRPGRTPSKLLGPPMPKEWESLEKSPKSTALRTKDIISLPLDRHESNHSIAAKNEGQAETQREAAWTKQGGPGRLCAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 34 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHRSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2004/0197875 A1
(43) Pub. Date: Oct. 7, 2004

(54) Modified cDNA factor VIII and its derivatives

(76) Inventors: Hans-Peter Hauser, Marburg (DE); Thomas Weimer, Gladenbach (DE); Martin Phillips, Wayne, PA (US)

Correspondence Address: Finnegan, Henderson, Farabow,, Garrett & Dunner, L.L.P., 1300 I Street, N.W., Washington, DC, 20005-3315, US

(21) Appl. No.: 10/721,997

(22) Filed: Nov. 26, 2003

(30) Foreign Application Priority Data
Nov. 29, 2002 (EP) 02026835.5

(51) Int. Cl. C12P 21/04; C07H 21/04; C07K 14/745

(52) U.S. Cl. 435/69.6; 435/320.1; 435/325; 530/384; 536/23.5

(57) ABSTRACT

Modified human factor VIII cDNA wherein mutations are inserted either in the wild-type factor VIII cDNA or in a factor VIII cDNA in which the B-domain is partially or completely deleted and may be replaced by a DNA linker segment and

A) one or several codons of the human factor VIII cDNA which are not identical with the corresponding codon in the same position of the porcine factor VIII cDNA are substituted by a different codon in such a way that

when the human sequence contains a codon for a neutral amino acid whereas the porcine sequence contains a codon for a charged amino acid then a codon for an amino acid with the same charge as found in the porcine sequence is introduced into the human sequence;

when the human sequence contains a codon for a charged amino acid whereas the porcine sequence contains a codon for a neutral amino acid then a codon for a neutral amino acid or a codon for an amino acid of the opposite charge is introduced into the human sequence,

when the human sequence contains a codon for a charged amino acid whereas the porcine sequence contains a codon for an amino acid with the opposite charge then a codon for an amino acid with the opposite charge is introduced into the human sequence or

B) one or several codons for a charged amino acid which are found in the FVIII cDNA of a hemophilic patient are replaced by a codon for an amino acid of the opposite charge.

The present invention relates to modified DNA sequences coding for biologically active recombinant human factor VIII and its derivatives with improved stability, recombinant expression vectors containing such DNA sequences, host cells transformed with such recombinant expression vectors, and processes for the manufacture of the recombinant human factor VIII and its derivatives. The invention also covers a transfer vector for use in human gene therapy which comprises such modified DNA sequences.

Classic hemophilia or hemophilia A is the most common of the inherited bleeding disorders. It results from a chromosome X-linked deficiency of blood coagulation factor VIII, and affects almost exclusively males with an incidence of between one and two individuals per 10.000. The X-chromosome defect is transmitted by female carriers who are not themselves hemophiliacs. The clinical manifestation of hemophilia A is an abnormal bleeding tendency and before treatment with factor VIII concentrates was introduced the mean life span for a person with severe hemophilia was less than 20 years. The use of concentrates of factor VIII from plasma has considerably improved the situation for the hemophilia patients. The mean life span has increased extensively, giving most of them the possibility to live a more or less normal life. However, there have been certain problems with the plasma derived concentrates and their use, the most serious of which have been the transmission of viruses. So far, viruses causing AIDS, hepatitis B, and non A non B hepatitis have hit the population seriously. Although different virus inactivation methods and new highly purified factor VIII concentrates have recently been developed an inadvertant contamination can not be excluded. Also, the factor VIII concentrates are fairly expensive because of the limited supply of human plasma raw material.

A factor VIII product derived from recombinant material is likely to solve a large extent of the problems associated with the use of plasma derived factor VIII concentrates for treatment for hemophilia A. However, the development of a recombinant factor VIII has met some difficulties, for instance the problem of achieving production levels in sufficiently high yields, in particular regarding the full-length molecule.

In fresh plasma prepared in the presence of protease inhibitors, factor VIII has been shown to have a molecular weight of 280 kDa and to be composed of two polypeptide chains of 200 kDa and 80 kDa, respectively (Andersson, L.-O., et al. (1986) Proc. Natl. Acad. Sci. USA 83, 2979-2983). These chains are held together by metal ion bridges. More or less proteolytically degraded forms of the factor VIII molecule can be found as active fragments in factor VIII material purified from commercial concentrates (Andersson, L.-O., et al. ibid.; Andersson, L.-O., et al. (1985) EP 0 197 901). The fragmented form of factor VIII having molecular weights from 260 kDa down to 170 kDa, consists of one heavy chain with a molecular weight ranging from 180 kDa down to 90 kDa, where all variants have identical amino termini, in combination with one 80 kDa light chain. The amino-terminal region of the heavy chain is identical to that of the single chain factor VIII polypeptide that can be deduced from the nucleotide sequence data of the factor VIII cDNA (Wood, W. I., et al. (1984) Nature 312, 330-336; Vehar, G. A., et al. (1984) Nature 312, 337-342).

The smallest active form of factor VIII with a molecular weight of 170 kDa, consisting of one 90 kDa and one 80 kDa chain, can be activated with thrombin to the same extent as the higher molecular weight forms, and thus represents an unactivated form. It has also been shown to have full biological activity in vivo as tested in hemophilia dogs (Brinkhous, K. M., et al. (1985) Proc. Natl. Acad. Sci. USA 82, 8752-8756). Thus, the haemostatic effectiveness of the 170 kDa form is the same as for the high molecular weight forms of factor VIII.

The fact that the middle heavily glycosylated region of the factor VIII polypeptide chain residing between amino acids Arg-740 and Glu-1649 does not seem to be necessary for full biological activity has prompted several researchers to attempt to produce derivatives of recombinant factor VIII lacking this region. This has been achieved by deleting a portion of the cDNA encoding the middle heavily glycosylated region of factor VIII either entirely or partially.

For example, J. J. Toole, et al, reported the construction and expression of factor VIII lacking amino acids 982 through 1562, and 760 through 1639 respectively (Proc. Natl. Acad. Sci. USA (1986) 83, 5939-5942). D. L. Eaton, et al. reported the construction and expression of factor VIII lacking amino acids 797 through 1562 (Biochemistry (1986) 25, 8343-8347). R. J. Kaufman described the expression of factor VIII lacking amino acids 741 through 1646 (PCT application No. WO 87/04187). N. Sarver, et al. reported the construction and expression of factor VIII lacking amino acids 747 through 1560 (DNA (1987) 6, 553-564). M. Pasek reported the construction and expression of factor VIII lacking amino acids 745 through 1562, and amino acids 741 through 1648, respectively (PCT application No. WO 88/00831). K.-D. Langner reported the construction and expression of factor VIII lacking amino acids 816 through 1598, and amino acids 741 through 1689, respectively (Behring Inst. Mitt., (1988) No. 82, 16-25, EP 0 295 597). P. Meulien, et al., reported the construction and expression of factor VIII lacking amino acids 868 through 1562, and amino acids 771 through 1666, respectively (Protein Engineering (1988) 2(4), 301-306, EP 0 303 540). When expressing these deleted forms of factor VIII cDNA in mammalian cells the production level is typically 10 times higher as compared to full-length factor VIII.

FVIII is secreted into plasma as a heterodimer of a heavy chain (domains A1-A2-B) and a light chain (A3-C1-C2) associated through a noncovalent divalent metal ion linkage between the A1- and A3 domains. In plasma, FVIII is stabilized by binding to von Willebrand factor.

Upon proteolytic activation by thrombin, FVIII is activated to a heterotrimer of 2 heavy chain fragments (A1, a 50 kDa fragment, and A2 a 43 kDa fragment) and the light chain (A3-C1-C2, a 73 kDa fragment). The active form of FVIII (FVIIIa) thus consists of an A1-subunit associated through the divalent metal ion linkage to a thrombin-cleaved A3-C1-C2 light chain and a free A2 subunit associated with the A1 domain. The dissociation of that free A2 subunit from the heterotrimer is thought to be the rate limiting step in FVIIIa inactivation after thrombin activation (Fay, P. J. et al, J. Biol. Chem. 266: 8957 (1991), Fay P J & Smudzin T M, J. Biol. Chem. 267: 13246-50 (1992)). The half life of FVIIIa in plasma is only 2.1 minutes (Saenko et al., Vox Sang. 83: 89-96 (2002)). To enhance the half life of FVIIIa would result into a longer acting FVIIIa which would also translate into less frequent injections of such a FVIII preparation. The inactivation of FVIIIa through activated Protein C (APC) by cleavage at Arg336 and Arg562 is thought not to be the rate limiting step. Attempts have been made to create a FVIIIa which is inactivation resistant by covalently attaching the A2 domain to the A3 domain and by mutating the APC cleavage sites (Pipe and Kaufman, PNAS, 94:11851-11856). However such a FVIIIa could have a thrombogenic potential as it is almost completely inactivation resistant. It is therefore the purpose of this invention to create a FVIIIa in which the A2 domain is stabilized without completely blocking inactivation.

FVIII is administered i.v. to haemophilia patients who are on prophylactic treatment about 3 times per week due to the plasma half life of FVIII of about 12 hours. It would thus be highly desirable to create a FVIII with enhanced plasma half life which could lead to a FVIII preparation which has to be administered less frequently. The present invention offers a solution to this problem by a modified FVIII molecule with an increased association of A2 to the A1/A3-C1-C2.

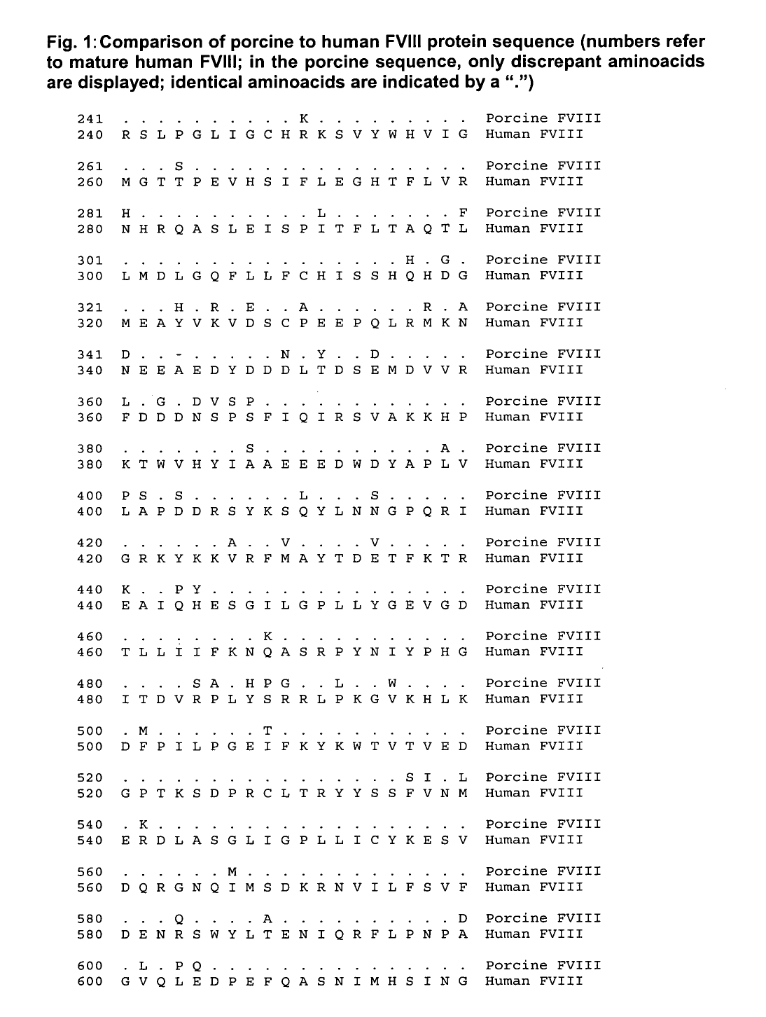

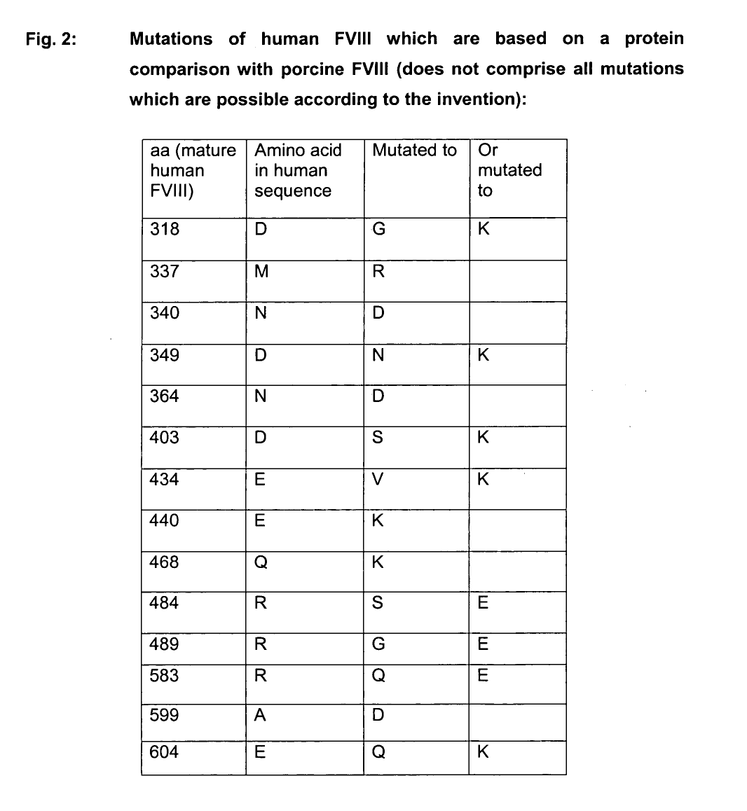

The nature of these modifications was identified by comparing the sequence of porcine FVIII to that of human FVIII as it is known that the dissociation of human A2 domain is threefold enhanced versus that of porcine A2 (Lollar et al., J Biol. Chem., 267:23652-23657 (1992)). The sequence comparison (FIG. 1) revealed several differences. A subset of these differences consists of differently charged amino acids. Mutants of human FVIII were constructed according to the following guidelines. When the human sequence contained a neutral amino acid whereas the porcine sequence contained a charged amino acid then a charged amino acid with the same charge as found in the porcine sequence was introduced into the human sequence. When the human sequence contained a charged amino acid whereas the porcine FVIII contained a neutral amino acid then a neutral amino acid or an amino acid of the opposite charge was introduced, e.g. if the human FVIII contained an acidic amino acid at a position where the porcine FVIII contained a neutral amino acid, also a basic amino acid was introduced. When the human sequence contained a charged sequence whereas the porcine FVIII contained a charged amino acid then an amino acid with the same charge as found in the porcine amino acid was introduced into the human sequence. Examples for such mutations which lead to an improved FVIII with a plasma half life of its activated form of more than three minutes, preferably of more than 10 minutes even more preferably more than 30 minutes, are listed in FIG. 2.

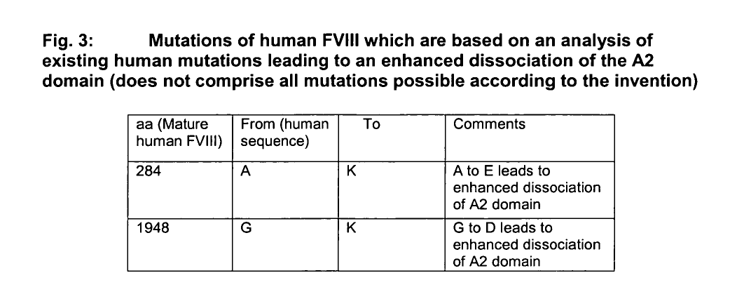

Other mutations for an improved FVIII were deduced by analyzing mutations in human FVIII which occurred naturally and which lead to a faster dissociation of the A2 domain associated with hemophilia. Such mutations result in differences between the two-stage assay as compared to the one-stage assay while determining FVIII clotting activity, whereas the two-stage assay result is lower than that of the one-stage assay as in the two-stage assay an incubation time of several minutes allows an unstable A2 domain to dissociate (Saenko et al., Vox Sang., 83:89-96 (2002). It was inferred that in those cases where such an increased instability was the result of the introduction of a charged amino acid that amino acid should be mutated into one of the opposite charge. Examples for such mutations which lead to an improved FVIII with a plasma half life of its activated form of more than three minutes, preferably of more than 10 minutes, even more preferably more than 30 minutes, are listed in FIG. 3.

As a basis for introducing the mutations preferably a modified factor VIII cDNA is used which comprises a first DNA segment coding for the amino acids 1 through 740 of the human factor VIII and a second DNA segment coding for the amino acids 1649 through 2332 of the human factor VIII. These two segments may be interconnected by a linker DNA segment, but the invention also encompasses introducing the mutations into full length FVIII.

Subject of the invention is therefore a modified human factor VIII cDNA wherein mutations are inserted either in the wild-type factor VIII cDNA or in a factor VIII cDNA in which the B-domain is partially or completely deleted and may be replaced by a DNA linker segment, and

A) one or several codons of the human factor VIII cDNA which are not identical with the corresponding codon in the same position of the porcine factor VIII cDNA are substituted by a different codon in such a way that

when the human sequence contains a codon for a neutral amino acid whereas the porcine sequence contains a codon for a charged amino acid then a codon for an amino acid with the same charge as found in the porcine sequence is introduced into the human sequence;

when the human sequence contains a codon for a charged amino acid whereas the porcine sequence contains a codon for a neutral amino acid then a codon for a neutral amino acid or a codon for an amino acid of the opposite charge is introduced into the human sequence

when the human sequence contains a codon for a charged amino acid whereas the porcine sequence contains a codon for an amino acid with the opposite charge then a codon for an amino acid with the opposite charge is introduced into the human sequence or

B) one or several codons for a charged amino acid which are found in the FVIII cDNA of a hemophilic patient are replaced by a codon for an amino acid of the opposite charge.

The production of factor VIII proteins at high levels in suitable host cells, requires the assembly of the above-mentioned modified factor VIII DNA's into efficient transcriptional units together with suitable regulatory elements in a recombinant expression vector, that can be propagated in E. coli according to methods known to those skilled in the art. Efficient transcriptional regulatory elements could be derived from viruses having animal cells as their natural hosts or from the chromosomal DNA of animal cells. Preferably, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA transcribed from the factor VIII DNA's, the transcriptional unit should contain in its 3′-proximal part a DNA region encoding a transcriptional termination-polyadenylation sequence. Preferably, this sequence is derived from the Simian Virus 40 early transcriptional region, the rabbit beta-globin gene, or the human tissue plasminogen activator gene.

The factor VIII cDNA's are then integrated into the genome into a suitable host cell line for expression of the factor VIII proteins. Preferably this cell line should be an animal cell-line of vertebrate origin in order to ensure correct folding, disulfide bond formation, asparagines-linked glycosylation and other post-translational modifications as well as secretion into the cultivation medium. Examples on other post-translational modifications are tyrosine O-sulfation, and proteolytic processing of the nascent polypeptide chain. Examples of cell lines that can be use are monkey COS-cells, mouse L-cells, mouse C127-cells, hamster BHK-21 cells, human embryonic kidney 293 cells, and preferentially CHO-cells.

The recombinant expression vector encoding the factor VIII cDNA's can be introduced into an animal cell line in several different ways. For instance, recombinant expression vectors can be created from vectors based on different animal viruses, Examples of these are vectors based on baculovirus, vaccinia virus, adenovirus, and preferably bovine papilloma virus.

The transcription units encoding the factor VIII DNA's can also be introduced into animal cells together with another recombinant gene which may function as a dominant selectable marker in these cells in order to facilitate the isolation of specific cell clones which have integrated the recombinant DNA into their genome. Examples of this type of dominant selectable marker genes are Tn5 aminoglycoside phosphotransferase, conferring resistance to Geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the factor VIII cDNA, or it can be encoded on a separate vector which is simultaneously introduced and integrated to the genome of the host cell, frequently resulting in a tight physical linkage between the different transcription units.

Other types of selectable marker genes which can be used together with the factor VIII DNA's are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthin, thymidin, and glycine. These dhfr-genes can be introduced together with the factor VIII cDNA transcriptional units into CHO-cells of the above type, either linked on the same vector or on different vectors, thus creating dhfr-positive cell lines producing recombinant factor VIII protein.

If the above cell lines are grown in the presence of the cytotoxic dhfr-inhibitor methotrexate, new cell lines resistant to methotrexate will emerge. These cell lines may produce recombinant factor VIII protein at an increased rate due to the amplified number of linked dhfr and factor VIII transcriptional units. When propagating these cell lines in increasing concentrations of methotrexate (1-10000 nM), new cell lines can be obtained which produce factor VIII protein at very high rate.

The above cell lines producing factor VIII protein can be grown on a large scale, either in suspension culture or on various solid supports. Examples of these supports are microcarriers based on dextran or collagen matrices, or solid supports in the form of hollow fibres or various ceramic materials. When grown in cell suspension culture or on microcarriers the culture of the above cell lines can be performed either as a bath culture or as a perfusion culture with continuous production of conditioned medium over extended periods of time. Thus, according to the present invention, the above cell lines are well suited for the development of an industrial process for the production of recombinant factor VIII that can be isolated from human plasma.

The recombinant factor VIII protein which accumulate in the medium of CHO-cells of the above type, can be concentrated and purified by a variety of biochemical and chromatographic methods, including methods utilizing differences in size, charge, hydrophobicity, solubility, specific affinity, etc. between the recombinant factor VIII protein and other substances in the cell cultivation medium.

An example of such a purification is the adsorption of the recombinant factor VIII protein to a monoclonal antibody which is immobilised on a solid support. After desorption, the factor VIII protein can be further purified by a variety of chromatographic techniques based on the above properties.

The recombinant proteins with factor VIII activity described in this invention can be formulated into pharmaceutical preparations for therapeutic use. The purified factor VIII proteins may be dissolved in conventional physiologically compatible aqueous buffer solutions to which there may be added, optionally, pharmaceutical adjuvants to provide pharmaceutical preparations.

The modified factor VIII DNA's of this invention may also be integrated into a transfer vector for use in the human gene therapy.

A further subject of this invention is a modified biologically active recombinant human factor VIII with improved plasma half life of its activated form wherein mutations are inserted either in the wild-type factor VIII or in a FVIII in which the B-domain is partially or completely deleted and replaced by a linker, and

A) one or several amino acids of the human factor VIII which are not identical with the corresponding amino acid in the same position of the porcine factor VIII are substituted by a different amino acid in such a way that

when the human sequence contains a neutral amino acid whereas the porcine sequence contains a charged amino acid then a charged amino acid with the same charge as found in the porcine sequence is introduced into the human sequence;

when the human sequence contains a charged amino acid whereas the porcine sequence contains a neutral amino acid then a neutral amino acid or an amino acid of the opposite charge is introduced into the human sequence;

when the human sequence contains a charged amino acid whereas the porcine sequence contains an amino acid with the opposite charge then an amino acid with the opposite charge is introduced into the human sequence or

B) one or several charged amino acids which are found in the FVIII amino sequence of hemophilic patients are replaced by a codon for an amino acid of the opposite charge.

The present invention will be further described more in detail in the following examples thereof. This description of specific embodiments of the invention will be made in conjunction with the appended figures.

Generation of FVIII mutants

For the generation of FVIII mutants, a suitable subfragment of the FVIII cDNA (e.g. Aval—Sacl, encompassing aminoacids 226 to 978) is first subcloned into a suitable cloning vector to reduce subsequent sequencing efforts. Site directed mutagenesis is then performed with a commercially available mutagenesis kit (e.g. QuickChange SiteDirected Mutagenesis Kit (Stratagene) according to the manufacturer's instructions. Primers used for mutagenesis are listed in the attached sequence listing and below, where the mutagenic bases are indicated in bold letters.

| | | |
|---|---|---|
| Mutation: A284K | | |
| Forward primer | ^{5′}GGAACCATCGCCAGAAGTCCTTGGAAATCTCGCC^{3′} | (Sequence 1) |
| Reverse primer | 5′GGCGAGATTTCCAAGGACTTCTGGCGATGGTTCC^{3′} | (Sequence 2) |
| Mutation: D318G | | |
| Forward primer | ^{5′}CCCACCAACATGGTGGCATGGAAGCTTATGTC^{3′} | (Sequence 3) |
| Reverse primer | ^{5′}GACATAAGCTTCCATGCCACCATGTTGGTGGG^{3′} | (Sequence 4) |
| Mutation: M337R | | |
| Forward primer | ^{5′}CAGAGGAACCCCAACTACGACGTAAAAATAATGAAGAAGCGGAAGAC^{3′} | (Sequence 5) |
| Reverse primer | ^{5′}GTCTTCCGCTTCTTCATTATTTTTACGTCGTAGTTGGGGTTCCTCTG^{3′} | (Sequence 6) |
| Mutation: N340D | | |
| Forward primer | ^{5′}CCCAACTACGAATGAAAAATGATGAAGAAGCGGAAGACTATG^{3′} | (Sequence 7) |
| Reverse primer | ^{5′}CATAGTCTTCCGCTTCTTCATCATTTTTCATTCGTAGTTGGG^{3′} | (Sequence 8) |
| Mutation: D349N | | |
| Forward primer | ^{5′}GAAGAAGCGGAAGACTATGATGATAATCTTACTGATTCTG^{3′} | (Sequence 9) |
| Reverse primer | ^{5′}CAGAATCAGTAAGATTATCATCATAGTCTTCCGCTTCTTC^{3′} | (Sequence 10) |
| Mutation: N364D | | |
| Forward primer | ^{5′}GGTCAGGTTTGATGATGACGACTCTCCTTCCTTTATCC^{3′} | (Sequence 11) |
| Reverse primer | ^{5′}GGATAAAGGAAGGAGAGTCGTCATCATCAAACCTGACC^{3′} | (Sequence 12) |
| Mutation: D403S | | |
| Forward primer | ^{5′}CCCTTAGTCCTCGCCCCCTCTGACAGAAGTTATAAAAG^{3′} | (Sequence 13) |
| Reverse primer | ^{5′}CTTTTATAACTTCTGTCAGAGGGGGCGAGGACTAAGGG^{3′} | (Sequence 14) |
| Mutation: E434V | | |
| Forward primer | ^{5′}GTCCGATTTATGGCATACACAGATGTTACCTTTAAGACTCG^{3′} | (Sequence 15) |
| Reverse primer | ^{5′}CGAGTCTTAAAGGTAACATCTGTGTATGCCATAAATCGGAC^{3′} | (Sequence 16) |
| Mutation: E440K | | |
| Forward primer | ^{5′}CCTTTAAGACTCGTAAAGCTATTCAGCATGAATCAGG^{3′} | (Sequence 17) |
| Reverse primer | ^{5′}CCTGATTCATGCTGAATAGCTTTACGAGTCTTAAAGG^{3′} | (Sequence 18) |
| Mutation: Q468K | | |
| Forward primer | ^{5′}CACACTGTTGATTATATTTAAGAATAAAGCAAGCAGACCATATAAC^{3′} | (Sequence 19) |
| Reverse primer | ^{5′}GTTATATGGTCTGCTTGCTTTATTCTTAAATATAATCAACAGTGTG^{3′} | (Sequence 20) |
| Mutation: R484S | | |
| Forward primer | ^{5′}CCCTCACGGAATCACTGATGTCTCTCCTTTGTATTCAAGG^{3′} | (Sequence 21) |
| Reverse primer | ^{5′}CCTTGAATACAAAGGAGAGACATCAGTGATTCCGTGAGGG^{3′} | (Sequence 22) |
| Mutation: R489G | | |
| Forward primer | ^{5′}GATGTCCGTCCTTTGTATTCAGGGAGATTACCAAAAGG^{3′} | (Sequence 23) |
| Reverse primer | ^{5′}CCTTTTGGTAATCTCCCTGAATACAAAGGACGGACAT^{3′} | (Sequence 24) |
| Mutation: R583Q | | |
| Forward primer | ^{5′}CTGTATTTGATGAGAACCAAAGCTGGTACCTCACAG^{3′} | (Sequence 25) |
| Reverse primer | ^{5′}CTGTGAGGTACCAGCTTTGGTTCTCATCAAATAGAG^{3′} | (Sequence 26) |
| Mutation: A599D | | |
| Forward primer | ^{5′}CTCCCCAATCCAGATGGAGTGCAGCTTGAG^{3′} | (Sequence 27) |
| Reverse primer | ^{5′}CTCAAGCTGCACTCCATCTGGATTGGGGAG^{3′} | (Sequence 28) |
| Mutation: E604Q | | |
| Forward primer | ^{5′}CAGCTGGAGTGCAGCTTCAGGATCCAGAGTTC^{3′} | (Sequence 29) |
| Reverse primer | ^{5′}GAACTCTGGATCCTGAAGCTGCACTCCAGCTG^{3′} | (Sequence 30) |
| Mutation: G1948K | | |
| Forward primer | ^{5′}CGATGGTATCTGCTCAGCATGAAGAGCAATGAAAACATCCATTCTATTC^{3′} | (Sequence 31) |
| Reverse primer | ^{5′}GAATAGAATGGATGTTTTCATTGCTCTTCATGCTGAGCAGATACCATCG^{3′} | (Sequence 32) |

After clone isolation and sequence verification mutant subfragments are reinserted into the respective expression vector.

Expression of FVIII Mutants

Transfection of FVIII mutant clones and expression of the mutant FVIII molecules is done as described previously and known to those skilled in the art (e.g. Plantier J L et al. Thromb. Haemost. 86:596-603 (2001)).

Measuring Affinity of A2 Subunit for A1/A3-C1-C2

The increased affinity of the A2 subunit for the A1/A3-C1-C2 can be measured as previously described by functional assays (Fay P J & Smudzin T M. J. Biol. Chem. 267:13246-50 (1992); Lollar P et al. J. Biol. Chem. 267:23652-57 (1992)) as well as a physical assay employing surface plasmon resonance (Persson E et al. Biochemistry 34:12775-81 (1995)).

The sequence of the porcine factor VIII is shown in Sequence 33, whereas the sequence of the human factor VIII is shown in sequence 34 of the attached sequence listing.

In the following Sequence Listing Sequences 1-32 describe oligonucleotides which are used to introduce specific mutations into FVIII. Sequence 33 is the amino acid sequence of full length mature porcine FVIII, Sequence 34 is the amino acid sequence of full length mature human FVIII.

1-10. (Canceled).

11. Modified human factor VIII cDNA, wherein at least one replacement of a first codon of wild-type human factor VIII cDNA is made, said first codon encoding a differently charged amino acid than the corresponding amino acid of the porcine factor VIII, and wherein said at least one replacement is chosen from:
(a) a first codon encoding a neutral amino acid is replaced with a second codon that encodes an amino acid with the same charge as the corresponding porcine factor VIII amino acid; and
(b) a first codon encoding a charged amino acid is replaced with a second codon that encodes an amino acid that is neutral or the same charge as the corresponding porcine factor VIII amino acid.

12. The modified human factor VIII cDNA of claim 11, wherein the B-domain is partially or completely deleted.

13. The modified human factor VIII cDNA of claim 12, wherein the deleted B-domain, or segment thereof, is replaced by a DNA linker segment.

14. The modified human factor VII cDNA of claim 11, further comprising at least one transcriptional regulatory element.

15. The modified human factor VII cDNA of claim 14, wherein the at least one transcriptional regulatory element is a dominant selectable marker.

16. A composition comprising the modified human factor VIII cDNA of claim 11 and a pharmaceutically acceptable carrier.

17. A recombinant vector comprising the modified human factor VIII cDNA of claim 11.

18. A recombinant host cell comprising the recombinant vector of claim 17.

19. A recombinant host cell comprising the modified human factor VIII cDNA of claim 11.

20. Modified human factor VIII cDNA, wherein at least one replacement of a first codon of a wild-type human factor VIII cDNA is made, said first codon encoding a differently charged amino acid than the corresponding amino acid of a mutant human factor VIII, and wherein said at least one replacement is:
(a) a first codon encoding a charged amino acid is replaced with a second codon that encodes an amino acid of the opposite charge as the corresponding mutant human factor VIII amino acid.

21. The modified human factor VIII cDNA of claim 20, wherein the B-domain is partially or completely deleted.

22. The modified human factor VIII cDNA of claim 21, wherein the deleted B-domain, or segment thereof, is replaced by a DNA linker segment.

23. The modified human factor VIII cDNA of claim 20, further comprising at least one transcriptional regulatory element.

24. The modified human factor VIII cDNA of claim 23, wherein the at least one transcriptional regulatory element is a dominant selectable marker.

25. A composition comprising the modified mutant human factor VIII cDNA of claim 20 and a pharmaceutically acceptable carrier.

26. A recombinant vector comprising the modified mutant human factor VIII cDNA of claim 20.

27. A recombinant host cell comprising the recombinant vector of claim 26.

28. A recombinant host cell comprising the modified mutant human factor VIII cDNA of claim 20.

29. A method of producing a modified human factor VIII protein, comprising:
culturing the host cell of claim 19 or 28 in cell suspension or on a solid support, as a bath cell culture or as a perfusion cell culture with continuous production of a conditioned medium; and
purifying said protein by chromatographic methods.

30. The modified factor VIII protein produced by the method of claim 29.

31. A method of treating hemophilia A, comprising administering the modified human factor VIII cDNA of claim 11 or 20 to at least one patient, and increasing or maintaining the plasma half-life of the activated, modified human factor VIII protein compared to wild-type human factor VIII protein.

32. The method of treating hemophilia A as claimed in claim 31, wherein the plasma half-life of the activated, modified human factor VIII protein is more than 3 minutes.

33. A method of treating hemophilia A, comprising administering the modified human factor VIII protein of claim 30 to at least one patient, and increasing or maintaining the plasma half-life of the activated, modified human factor VIII protein compared to wild-type human factor VIII protein.

34. The method of treating hemophilia A as claimed in claim 33, wherein the plasma half-life of the activated, modified human factor VIII protein is more than 3 minutes.

35. A modified human factor VIII protein comprising at least one mutation selected from the group consisting of A284K, D318G, M337R, N340D, D349N, N364D, D403S, E434V, E440K, Q468K, R484S, R489G, R583Q, A599 D, E604Q, and G1948K.
